SEQUENCE LISTING (26 sequences)

SEQ ID NO: 1 - DNA, 5040 bp, Homo sapiens
  variation (3687) .. (3687): single nucleotide polymorphism at position 3687 of NCB I Accession Number NG 009031 (A3687G)
tccatttttatgttactatttccaggaaaaatttaaaatagccttcatatacaataaaagcaagttagataaattatattgtacttatacaaataattaatacataggcatataaatgaagttaaaaaaatattcgatagcattattttggaaaaacttaagataggttatatgaaagtaggctaccaaagactatgtatgatgtatttccaaattagcttgaagcaaaaggtacagttgtgcatgagaataagcaattaaaaaaaagaaaaagcatatatatatatatggtagcacaaaaatgtccttagtatccaagctccaggactttgcctcttagtgtgtagtttgtgaactccctctatcagcttcacagaggacagtagtataacgctgtcctcaagtctactgaaaccaaagcccagggatgtggcctagaaatctggatgtttaaatagcactttgatgaatttgataatgtgttactttgcaatttttctgagagtgagactccaaagatagtcaagggtttgggaagagtaaatttaagccaattcagtaaattaggtgataaacaaagaagcttgctgtttttctctctttttttatgttttaaaacaatgagtttctccttttttgctcaagccctgaacaatttagttcagaatttgttttcttactttgattctgctggctttgccaattccatgaaaatccacttggcaccacatttccttagaagctcttgctttttcttgattttctttctgttcttagagtctaatattctagtgtctcaccaaattgttgttgtctctttccaaaactcctttaaccaaactatctgtatcttcatgtaaaaggcaccacagacctcccatcactcactctcccttaaccccaaaatatctatgtgttatcccttacagatgtcacttcctgcaaggccttccatattagttgctacaacatcactaaagaattttattacctgtggctctggacctacttctaatatctctggagatatccaccttcaaacacctgccacatccagctatgagggtctagtacctgggctccagagacatgttcaggttagttctgattataagaacatttctcttttttgccagaattgttggaaagttatctctattcaacagtctgtgtaggatttcagaagattgaatatacatataaatatatatacatacacacatgtatatatacacacacacatatatgtatatgtactttgtccatgtcgtatttggttattccaattctgggaaaataaattaagaaaataattaaaagatgcactcaaatatcgatatacaaaatgcctattgtaataatacataaaaaaagtcagaagctaacctcggtatctgtatgatttggagcaagttacttaaactctttgtggctatattttctcaaatgtaaaataaggttaataatagtgcttattttaaaggtagtcatgagttttaaatgaattcatgtattcaaagtggatagaatagtgctttcaaataatatttattacctaaaattattattatcatcatataagtaacaagagatacaatgaaccataatatgcccatatgataatatatcacacagtaatcaaaatattttaataggaaattaatatcataagataaaaagcaagaagaatgatgataagccatttgactaccactatatttatctatgtaactataaccacatacacatattttaaaaaattgattggaatatacaaaatggtcttttcattctctgtgatatgtaaaatgtagaaatatacttttctgggctggatgcagtagctcacacctataatcccagcactttgggaggccgaggttggtggagcacctgaggtcaggaattcgagaccagcctggccaacatagtgaaatcctgtgtctactaaaaatacaaaaattagccagatttggtggtgggtgcctgtaatcccagctactcaggaggctgaggcaggaaaattgcttgaacccaggaggcagaggttgcagtgagccgagatcctgccactgcactccagcctgggcgacagagtgagactccatctcaggaaaaaaaaaaaaaaatctatctatctatctatctatctatctatctatctatctatctatctatctatacttatatatacttttctttgttatccatattcatttcagagaatgcagtgctattgtaaaaaaaatgaataaaattaatataagtttagagcacttttttggcatcagtatgcctaaggaaaactaatgtataatacaggattaatgtttttcacaccatctcaaatcttactgaatgctgctatctagaaaacagtggtaactgagttacaatgggaccttaaaagcaaaaaaccactgtgactggctaactgaaagtgatcatagagccaaaaccataatgattgtcctgcccaagcttcagcatatatggttgcaaaatgtgcgcagtgttaatgccgtgtcaagtgtcttgtggtatttcatcagtgtcgcttacaaggcatactcaacatttaacagcaggatcattaaccatccatgctgtgatgtaatggaatagtgcttccctactaaccctgaaacaagctcactgtgtgtgacctacatccttccgctggatgagtgtgagaaaatgatgacaccacagaagcaaagcagctgctgtaagttgagccacaaggaagaaactctcaagacaggaagaataaatgctttagaggtgcactaaaataatgaatgaactaatgtggcatagctgtgaaatcatgcgaggaaaagaactacagggacagtaacagaaggtggttctttgaagtaaagacattgagaagaagaaaaattgccaacttgtgtaatcagttgatgttgctagttagccagtttaaacatgcgttaaagataaagatctgcttgaaaccaattattttattgtttggcttcctgtttaaagatgaaaatgaaatatgtgtatatatgtgacatattttacctagcagtgagtagatgcaggaagcatgtaaaatgctttgctgaattcactaacgtcacaaaatattccttaataattgccgcgtttgataggctgaccttatgctagcacaggaaacaatgcagtggacttgggttggagagaagaattttgacatttggatttttaaaaatcatgtatattgaacccatgaactctcatgtggaacatcagatttactatattttaaacagaaatgccatcactataaaaaccattgcaaatttgtcaatatacttccatttaaaaagccttttcctgcttttattgatttattgattaatttttttttgagacaggatctcactctgtcacccacgatgaagtgcagtggcacaatcgtggcttgcagcctcgacctcctaggctcaggtgatactcttacctcagctacctcccaagtaactgggaatgtaggcttgcaccaccatgtctgacccattttcttgcagaaattgagttttgctatgttgtccagacttgtcttgaactcccagactgaagcaatccgcccacttaggcctcctaaaatgttgggattacagtcatgaaccactgtgctccacctcctttttctccttttcagatagattatgtattctgtaccttttgtgtaactaacttgttattacataaactatattctaaatacattataaattattgtgatcatgatatttatcattgaattaacactgcctaagtagcaattttcacagaaaagataattttaaaagaccaaaatggttattctcttggaagcagttgacataaaataacagaataatacagattaaaattctgttttccaacagctactagtggtaagaaatatagtaagcttctgatttgtagaaaccttagctttctcatctgatgaggtggttaataatattaactctcatatttgctgaaataatataaaatatttaatataaattgtcaattataatgactggcataaggtagaattacaacaaatgctaattatatttgcttgtttgaaaatttaacactctattaaaaggcaaaaaagtgctcacctgcaaatattaatacatgccttctgtttagtccatagaaagcccacaagtaagagattcaaaagtgaaagctatcacttacagaagattaagaatatattgcattgagaagatagggattaaggagtttatcaaaggttttttaaaaaattattttcctgaacatcatttcaagaaagataagcgccaaagacataaatatattctccagagcatgtgcactgcaagttgacaaatgtgtctcttttatagagtttggtgaaggttattcattttatttttcctgtcttgaccagaaaattgtggcttctcatcatgtatgactgcctgcagggtcttctgaataggaaattaccaatgctgaaatttgtatgacttaaataagtgttgaagtgtgggggttgaaaggagatacatatcagagacatccattcagttaatagaactaagtttttgatttgcatagcttatatctagagcattgtgtttcttattttctatattactatcttctctaactatataattagcacagattgaagctataatggatttcaataatagcactactttagaatgatgtatacgtctttaaatcttaagacaagcataggaaatatgtatattcttaaaaaaaggaaaatttttttaaagtttagagttaaaagaaacatgcattttctgttacatttcagattattttagttaaagaatcttctatgctttttttcttcattcttagaagactacctgcaattgtaaagcatgaatcctgtaatacttctgtaaatgaatcactattgcatttataccattagcctctgaacagatttcaagttgctgctgccaactctcgcgagctttgtcagtaacagttgattgttacattcagtaacactgaatg

SEQ ID NO: 2 - DNA, 20 bp, Artificial Sequence
  Primer for BChE promoter sequencing BChEproml UA
tgataggctgaccgtatgct

SEQ ID NO: 3 - DNA, 20 bp, Artificial Sequence
  Primer for BChE promoter sequencing BChEpromlL
acctcatcagatgagaaagc

SEQ ID NO: 4 - DNA, 21 bp, Artificial Sequence
  Primer for BChE promoter sequencing BChEprom2UA
tctcttggaagcagttgacat

SEQ ID NO: 5 - DNA, 22 bp, Artificial Sequence
  Primer for BChE promoter sequencing BChEpromS6
ccattatagcttcaatctgtgc

SEQ ID NO: 6 - DNA, 24 bp, Artificial Sequence
  Primer for BChE promoter sequencing BChEprom2UB
agatacatatcagagacatccatt

SEQ ID NO: 7 - DNA, 20 bp, Artificial Sequence
  Primer for BChE promoter sequencing BChEprom2L
gaagagatcactctcatccc

SEQ ID NO: 8 - DNA, 23 bp, Artificial Sequence
  Primer for genotyping A-1314G BChE -1314U
tcttgaactcccagactgaagca

SEQ ID NO: 9 - DNA, 23 bp, Artificial Sequence
  Primer for genotyping A-1314G BChE -1314G
tacacaaaaggtacagaatacac

SEQ ID NO: 10 - DNA, 43 bp, Artificial Sequence
  Primer for genotyping A-1314G BChE -1314A
ttatgtaataacaagttagttacacaaaaggtacagaatacat

SEQ ID NO: 11 - DNA, 23 bp, Artificial Sequence
  Primer for genotyping A-795G BChE -795U
aagtgctccacctgcaaatatta

SEQ ID NO: 12 - DNA, 22 bp, Artificial Sequence
  Primer for genotyping A-795G BChE -795G
taatcttctgtaagtgatagcc

SEQ ID NO: 13 - DNA, 42 bp, Artificial Sequence
  Primer for genotyping A-795G BChE -795A
ttctcaatgcaatatattcttaatcttctgtaagtgatagct

SEQ ID NO: 14 - DNA, 22 bp, Artificial Sequence
  Primer for genotyping C-558T BChE -558L
tgtctctgatatgtatctcctt

SEQ ID NO: 15 - DNA, 21 bp, Artificial Sequence
  Primer for genotyping C-558T BChE -558CS
tcttgaccagaaaattgtggc

SEQ ID NO: 16 - DNA, 42 bp, Artificial Sequence
  Primer for genotyping C-558T BChE -558TL
tattcattttatttttcctgtcttgaccagaaaatttgtggt

SEQ ID NO: 17 - DNA, 27 bp, Artificial Sequence
  Primer for genotyping BchE-K BchE-4U
ctgtactgtgtagttagagaaattggc

SEQ ID NO: 18 - DNA, 30 bp, Artificial Sequence
  Primer for genotyping BchE-K BchE-K
atggaatcctgctttccactcccattccgt

SEQ ID NO: 19 - DNA, 50 bp, Artificial Sequence
  Primer for genotyping BchE-K BchE-W
atcatgtaattgttccagcgtaggaatcctgctttccactcccattctcc

SEQ ID NO: 20 - DNA, 22 bp, Artificial Sequence
  Primer BChE 2U for real time PCR
gagtagatccatagtgaaacgg

SEQ ID NO: 21 - DNA, 20 bp, Artificial Sequence
  Primer BChE 6LRNA for real time PCR
cagcgatggaatcctgcttt

SEQ ID NO: 22 - DNA, 20 bp, Artificial Sequence
  Primer beta-actin U2 for real time peR
tctacaatgagctgcgtgtg

SEQ ID NO: 23 - DNA, 20 bp, Artificial Sequence
  Primer beta-actin L3 for real time peR
tagatgggcacagtgtgggt

SEQ ID NO: 24 - DNA, 20 bp, Artificial Sequence
  Primer GUS-Ul for real time peT
atgtggttggagagctcatt

SEQ ID NO: 25 - DNA, 20 bp, Artificial Sequence
  Primer GUS-L2 for real time peR
tgtctctgccgagtgaagat

SEQ ID NO: 26 - DNA, 1980 bp, Homo sapiens
  variation (934) .. (934): single nucleotide polymorphism corresponding to position 68974 of the NCBI Accession Number NG 009031 (G68974A). It is the K variant polymorphism. This SEQ ID NO 28 represents the region from position 68041 to 70020 of this Accession Number.
ggaggcagaggttgcggtgagccaagatggcgccattgcactccagcctgggtggcaagagtgaactctgtctcaaaaaaaaataaataaataaaataaaacattgtattataataagggtaaaatctatgtggaatgataattaaaaagttatccttatgcattaataaatacagtatgacctttgtatttgcgggttacacatccatggatttaaccaaccacgagtcaaaaacatttgataaataaatacatctatactaaacatatacagacctttttttcttatcattattccttaaacaatataatataataaacatttacacagcactttcattatattaggtatgaattatctagggatgtgcatggcttatgtgaaaatgttatgctattttacattagggacttgagtatgagaggattttggtatccctgagaggtcctagaaccaactccccacaaatactaaggatgactgtagtcatatataattttcatcagaaataattatctgttatataactatatatgtaaatgagaccattaaatgaaatttgtgttattttaaagtgggtcaagaaaagagcataatatctacatatcctcttgaaaactaaagaaaatatatgtaatgtaattatacataatacaatatataaactcattttccattttatatgttatacgttatataaattatatatacattatttgcattttcacataatctgtaaagattttattaaaatctcttttcaggcaaagcgagctaataacaaataataaagaataaataaagaaaataatgctgtactgtgtagttagagaaaatggcttttgtattcgaaattatttttcagttaatgaaacagataaaaattttgattaatacaacttattccatattttacaggaaatattgatgaagcagaatgggagtggaaagcaggattccatcgctggaacaattacatgatggactggaaaaatcaatttaacgattacactagcaagaaagaaagttgtgtgggtctctaattaatagatttaccctttatagaacatattttcctttagatcaaggcaaaaatatcaggagcttttttacacacctactaaaaaagttattatgtagctgaaacaaaaatgccagaaggataatattgattcctcacatctttaacttagtattttacctagcatttcaaaacccaaatggctagaacgtgtttaattaaatttcacaatataaagttctacagttaattatgtgcatattaaaacaatggcctggttcaatttctttctttccttaataaatttaagtttttccccccaaaattatcagtgctctgcttttagtcacgtgtattttcattaccactcgtaaaaaggtatcttttttaaatgaattaaatattgaaacactgtacaccatagtttacaatattatgtttcctaattaaaataagaattgaatgtcaatatgagatattaaaataagcacagaaaatcattggtctctttgttttttatataaaggcaagaaagaatcttaaaatgagaggctcagccactttaaaacaccttataatagaaacagaatgctgctaccaagaaacaaaatataaaataatgtaattatttatgtaatgccttcaaattatattcttcatttgactcttgctctccctcacctcctctatcctcaaatcaccacaaacatgtacatatgtgaattacctttccgtaattcaaaaatttttaaaaagtgggtgcatagaagtctaaagttaggttgtgtaccatctgggacagggcaacccttgcctggacccagaaatattaacatattaataggaaaattgttcaattccagaagagaaatggttgtactttctgtagacaaactgtcttatttttgcaattaaaaaaattacagagctgcatttattagcatgcttggcaagggag

(19) United States
(12) Patent Application Publication
(10) Pub. No.: US 2014/0249047 A1
(43) Pub. Date: Sep. 4, 2014

(54) GENETIC MARKER FOR THE DIAGNOSIS OF DEMENTIA WITH LEWY BODIES

(71) Applicants: Katrin Beyer, Badalona (ES); Montserrat Domingo, Badalona (ES); Aurelio Ariza, Badalona (ES)

(72) Inventors: Katrin Beyer, Badalona (ES); Montserrat Domingo, Badalona (ES); Aurelio Ariza, Badalona (ES)

(73) Assignee: UNIVERSITAT AUTONOMA DE BARCELONA, Ceranyola del Valles (ES)

(21) Appl. No.: 14/236,054

(22) PCT Filed: Jul. 30, 2012
(86) PCT No.: PCT/EP2012/064871

§ 371 Date: May 9, 2014

(30) Foreign Application Priority Data
Jul. 29, 2011 (EP) 11006277.5

(51) Int. Cl. C12Q 1/68

(52) U.S. Cl. CPC C12Q 1/6883; C12Q 2539/10
USPC 506/9; 435/6.11; 536/24.33

(57) ABSTRACT

Specific polymorphisms in BChE gene have been found which allow determining whether a patient suffers from dementia with Lewy bodies (DLB), and allow distinguishing it from Alzheimer's disease. The invention provides an in vitro method for the diagnosis of DLB comprising determining in a biological sample from a subject, the genotype of the following polymorphisms in butyrylcholinesterase (BChE) gene: the polymorphic site at position 3687 in NCBI Accession Number NG_009031 (i.e. SEQ ID NO: 1) the polymorphic site at position 4206 in SEQ ID NO: 1, the polymorphic site at position 4443 in SEQ ID NO: 1. and the polymorphic site at position 68974 in NCBI Accession Number NG_009031 (i.e. position 934 in SEQ ID NO: 26).

The present invention relates to the field of medicine, and particularly to neurodegenerative disorders. It specifically relates to markers for the diagnosis of dementia with Lewy bodies.

### BACKGROUND ART

Lewy body diseases comprise a group of disorders characterized by the presence of proteinaceous neuronal inclusions called Lewy bodies (LB). Clinically, two disorders can be distinguished: Parkinson disease (PD) and dementia with Lewy bodies (DLB). Whereas PD is the most common progressive movement disorder in the elderly, DLB is the second most frequent cause of dementia after Alzheimer disease (AD). While widespread distribution of LB in virtually every brain area is a typical feature of DLB, the substancia nigra is the most affected in PD.

When first described, DLB was thought to be an infrequent disorder, but over the last years intense investigation has revealed that it accounts for 10-15% of autopsied cases. Main DLB symptoms include fluctuating cognitive impairment, recurrent visual hallucinations and Parkinsonism, but nevertheless, many AD overlapping symptoms lead to a frequent misdiagnosis of DLB. Since AD and DLB patients may differ in terms of response to medication and prognosis, it is important to improve accuracy in diagnosing DLB.

To achieve a better clinical distinction between DLB and AD, various longitudinal and comparative studies have been carried out during the last years. Patients with only Lewy body (LB) pathology show relatively less severe impairments but more pronounced deterioration of executive function and attention than patients with only AD or mixed AD/LB pathology. Moreover, DLB patients exhibit a slower decline of recognition memory but have more psychiatric symptoms than patients with AD, where this kind of symptomathology is observed at later disease stages. Finally, the presence of visual hallucinations in early-stage dementia has been shown to be most specific for DLB. It is noteworthy to mention that although a high specificity (ranging from 90 to 99% in different studies) of clinical diagnosis is achieved, its sensitivity remains relatively low (18-83%). Accordingly, the first consensus guidelines established in 1996 for the clinical diagnosis of probable and possible DLB have been revised to improve the sensitivity for DLB diagnosis, but nevertheless, many AD overlapping symptoms lead to a frequent misdiagnosis of DLB between 40-80% of the cases.

The main cause of low diagnostic sensitivity for DLB comes from the elevated percentage of cases that show in addition to LB related pathology AD characteristic changes. To assess this type of combined pathology, the third DLB consortium proposed a model to place AD-related pathology into the context of LB pathology. The higher the stage of AD-type pathology the lower is the sensitivity to achieve a correct diagnosis of DLB. Accordingly, a recent report confirmed that the misdiagnosis of DLB increases with increasing AD associated pathology, but even so, only around 52% of patients had received the correct diagnosis of DLB at low AD-pathology stages.

The treatment of DLB is symptomatic and is based on a limited number of clinical trials and extension of results from trials in AD. At the moment AD treatment consists of using cholinesterase inhibitors to improve the effectiveness of acetylcholine either by increasing the levels in the brain or by strengthening the way nerve cells to respond to it. Moreover, neuroleptic drugs are used to diminish psychotic symptoms normally present during the disease course. On the contrary, for treating DLB the use of neuroleptics may cause adverse reaction in about 50% of DLB patients and may cause death.

Thus, the ability to differentially diagnose between AD and DLB will be a major advantage not only for the individual patient being treated, but also with respect to the economic strains of public health systems. However, at present, precise differentiation of AD and DLB is only possible by post-mortem analysis of brain tissue.

Nowadays, diagnosis of DLB is based on clinical evaluation of symptoms and traits, following the guidelines established by the Consortium on DLB International Workshop (I. G. McKeith, “Consensus guidelines for the clinical and pathologic diagnosis of dementia with Lewy bodies (DLB): report of the Consortium on DLB International Workshop”, J. Alzheimer's Dis. 2006, vol. 9, pp. 417-23), but as explained above, it leads to misdiagnosis of DLB. Image methods like positron tomography (PET) and single photon emission computer tomography (SPECT) are available, but their sensitivity is not very high and they are very expensive for a routine clinical use. An early unequivocal diagnosis would give a therapeutic margin to reduce or stop the disease progression.

There have been some attempts in trying to find genetic markers to precisely identify a patient with DLB. A genetic test would be a very useful tool and easy to perform in the daily clinical practice in the pre-mortem diagnosis of DLB. In this sense, some proteins and genes studied in order to find a relationship with DLB are alpha-7 nicotinic acetylcholine receptor subunit, osteopontin, nitric oxide synthase, ubiquitin carboxy-terminal hydrolase L1 gene, BDNF gene, or beta-synuclein gene. Many of them have been studied in brain samples at an experimental level and they are not useful in real clinical diagnosis because of the difficulties to obtain a patient brain biopsy.

Butyrylcholinesterase (BChE) is a glycoprotein enzyme synthesized in the liver. In the human brain it is found principally in glia, particularly in cortical and subcortical structures, but it is also found in neurons above all, those implicated in cognitive functions. In AD patients BChE is found in amyloid plaques, as well as, in neurofibrillary tangles. This enzyme acts as a detoxification enzyme of organophosphorus and carbamate compounds and hydrolyzes succinylcholine, aspirin and cocaine. BChE function in the human brain is not well known, but it is known that hydrolyzes acetylcholine (ACh) when acetylcholinesterase (AChE) is reduced or absent. It is a marker for determining apnea susceptibility. Up to the moment 65 variants have been identified in BChE gene which is located in chromosome 3 (3q26.1-q26.2) (cf. F. Parmo-Folloni et al., “Two new mutations of the human BCHE gene (IVS3-14T>C and L574fsX576)” Chemico-Biological Interactions 2008, vol. 175, pp. 135-7).

The presence of mutation A539T in exon 4 of BChE gene is named K variant in honor of Werner Kalow. The K-variant is associated with a DNA transition from guanine to adenine at nucleotide 1615 in the mRNA corresponding to position 68974 in the DNA sequence (NCBI Accession Number NG_{—}009031), which causes an amino acid change from alanine 539 to threonine.

The K-variant is situated at the C-terminal of the protein, responsible for its tetramerization on one hand, and for the attenuation of beta-amyloid fibril formation, on the other. In serum the BChE K variant is responsible for a one third reduction of serum BChE activity levels. Although main BChE functions in brain remain unknown, the K-variant seems to diminish the rate of attenuation of beta-amyloid fibril formation, accelerating AD progression. On the contrary, tau protein is less phosphorylated in AD patients that carry at least one K-allele, representing a protective mechanism for AD.

Many studies have investigated a possible association between BChE gene, specially the BChE K variant, and AD. Co-occurrence of the epsilon 4 allele of the apolipoprotein E gene (ApoE4), the major known genetic risk factor for AD, and BChE gene variants have been discussed to influence AD pathology. Some reports show an increased risk for AD in subjects with a combination of BChE wild type and ApoE4 genotype. Others found that the combination of BChE K and the ApoE4 increased the risk for AD. The progression of cognitive decline in AD has been shown to be influenced by the BChE genotype. However, there is not a definitive conclusion about the role of BChE K variant as neither a risk factor nor a progression marker for AD.

The possible association of BChE K genotype and DLB has also been studied. Singleton et al. (A. B. Singleton et al., “Butyrylcholinesterase K: an association with dementia with Lewy bodies”, Lancet 1998, vol. 351, pp. 1818) reported an increased frequency of homozygous BChE K carriers in DLB compared to controls. A recent study found increased BChE K and ApoE4 frequencies in DLB patients compared to PDD patients (R. Lane et al., “BuChE-K and APOE epsilon4 allele frequencies in Lewy body dementias, and influence of genotype and hyperhomocysteinemia on cognitive decline”, Mov. Disord. 2009, vol. 24, pp. 392-400). Based on the hypothesis that a higher percentage of DLB than PDD subjects have additional AD-type pathology, and additional AD type pathology leads to more rapid cognitive decline, the authors concluded that this genotype may be important in dementia onset and progression in LBD. However, a recent study shows that there is not a significant association between the BChE K variant and the demented DLB phenotype (cf. W. Maetzler et al., “No differences of butyrylcholinesterase protein activity and allele frequency in Lewy body diseases” Neurobiol. Dis. 2009, vol. 35, pp. 296-301).

Therefore, there is the need of providing means for an accurate identification of a patient suffering from dementia of Lewy bodies, and distinguishing from Alzheimer disease, to be used in the common clinical practice.

### SUMMARY OF THE INVENTION

The inventors have found specific polymorphisms in BChE gene which allow determining whether a patient suffers from dementia with Lewy bodies, and distinguishing it from Alzheimer disease.

There are documents in the state of the art that intend to find an association between BChE K variant and DLB, but as explained below, recent studies consider that there is no significant association (cf. W. Maetzler et al., supra). Surprisingly, the inventors of the present invention have found that specific information for diagnosis of DLB is obtained with genotype of K variant in cooccurrence with the genotype of three further polymorphisms in BChE gene. Determining these genotypes is therefore useful to distinguish DLB from AD, these two genotypes constituting a specific genetic marker for DLB.

Thus, the inventors have observed that a combination of genotypes gives rise to identify a group of patients suffering from DLB, and distinguishing from AD. This combination is formed by the genotypes of the polymorphic sites at positions 3687, 4206, and 4443 in NCBI Accession Number NG_{—}009031 (i.e. positions 3687, 4206, and 4443 respectively in SEQ ID NO: 1), and the polymorphic site at position 68974 in NCBI Accession Number NG_{—}009031 (i.e. position 934 in SEQ ID NO: 26).

Positions of the polymorphisms in BChE nucleotide sequence are given from the nucleotide sequence of NCBI Accession Number NG_{—}009031 which corresponds to the promoter and the gene. This sequence was published on 31 Jan. 2010.

The polymorphic sites 3687, 4206, 4443 are in the promoter region. For these sites, reference is made also to the SEQ ID NO: 1, which corresponds to the sequence from nucleotide 1 to nucleotide 5040 of the complete sequence of BChE at NCBI. A possible numbering of the nucleotides sometimes used takes the transcription start as position 1 and consequently, the nucleotides upstream this position as negative positions. Transcription start position 1 corresponds to position 5001 in NG_{—}0090031. The correspondence between the numbering used in this description and the “negative” one, is given herein:

A3687G corresponds to A-1314G
A4206G corresponds to A-795G
C4443T corresponds to C-558T

The polymorphic site at position 68974 is in the codifying region of NG_{—}009031. The region from position 68041 to 70020 of NG_{—}009031 is included as SEQ ID NO: 26. Taking this region alone, the nucleotides are renumbered, so consequently, the position 68974 in the complete gene sequence becomes the position 934 in SEQ ID NO: 26. This polymorphism is associated to the change of amino acid in exon 4 of BChE resulting in the K variant. The position also used in the literature for this polymorphism is 1615 due to a different sequence numbering (with reference to the mRNA sequence which codifies for the mature BChE protein, without the signal peptide).

As described in the examples below, no specific association has been found between each of the four polymorphisms in BChE gene independently evaluated and DLB; but surprisingly, these polymorphisms in combination give specific information for DLB.

Accordingly, an aspect of the invention provides an in vitro method for the diagnosis of DLB comprising determining in a biological sample from a subject, the genotype of the following polymorphisms in butyrylcholinesterase (BChE) gene: the polymorphic site at position 3687 in NCBI Accession Number NG_{—}009031 (i.e. SEQ ID NO: 1), the polymorphic site at position 4206 in SEQ ID NO: 1, the polymorphic site at position 4443 in SEQ ID NO: 1, and the polymorphic site at position 68974 in NCBI Accession Number NG_{—}009031 (i.e. position 934 in SEQ ID NO: 26).

As it is shown in the examples below, post mortem samples of AD (n=26), pure DLB (n=12), common DLB (n=24) and controls (n=23) were analyzed, as well as clinically diagnosed samples obtained from 223 AD and 160 control subjects. As a result, two relevant genotypic combinations are described.

One of the genetic markers is the genotype combination, AAAGCCK+. It is constituted by the specific genotypes of the polymorphic sites at positions 3687 (both alleles contain an adenine at this position), 4206 (one allele contains an adenine and the other a guanine), 4443 (both alleles contain a cytosine), and 68974 (at least one of the two alleles contains an adenine). The determination of this genotype combination in demented patients serves as differential diagnostic marker providing the clinical diagnosis of DLB but it may also serve as early diagnostic marker for DLB in asymptomatic individuals.

In another embodiment, the invention relates to a genetic marker which is a genotype combination, AAAAC+KW. It is constituted by the specific genotypes of the polymorphic sites at position 3687 (both alleles contain adenine at this position), 4206 (both alleles contain adenine at this position), 4443 (at least one of the two alleles contains a cytosine at this position), and 68974 (one allele contains an adenine and the other guanine). The determination of this genotype combination in demented patients serves as differential diagnostic marker providing the clinical diagnosis of DLB, but it may also serve as early diagnostic marker for DLB in asymptomatic individuals.

Advantageously, within the great heterogeneity of DLB and according to the examples, the method of the invention allows to differentially detect the 30-60% of DLB cases, which otherwise would be diagnosed as AD. This percentage of patients, difficult to diagnose in the clinical practice, will receive the correct diagnostic from the beginning of the disease. The specificity for the disease is of 96.8%. This represents a first specific marker for DLB.

Until now, the available tools in the state of the art did not allow the specific identification of DLB in the clinical practice. In this way, when the subject was diagnosed of AD, he was submitted to therapy with neuroleptics, which is the most adequate treatment for psychotic symptoms in AD but more than 50% of DLB patients exhibit an adverse reaction to this kind of treatment causing death in many cases. The method of the invention is of importance because it will enable the medical community to apply adequate treatment to patients suffering from DLB without the risks of an incorrect therapy. Therefore, applying the method of the invention, diagnostic specificity for DLB is increased as well as deaths caused by adverse effects of treatment with neuroleptics will be reduced.

Furthermore, as the method of the invention allows to specifically diagnosing patients with DLB, is it possible to have a defined group of patients to be included in a clinical trial.

By “diagnosis” in medicine it is meant the act or process of recognition of a disease or condition by its outward signs, symptoms, and underlying physiological/biochemical cause(s).

By “determining the genotype” in this description it is meant identifying the nucleotide in a given position.

In this description “a given nucleotide in one allele” means that the subject is heterozygote for that nucleotide in that gene, and “in both alleles”, which is homozygote for that nucleotide.

According to the invention, the method includes determining the polymorphisms indicated on BChE gene, but also determining polymorphisms in linkage disequilibrium with said polymorphisms which would give the same information. In population genetics, linkage disequilibrium is the non-random association of alleles at two or more loci, not necessarily on the same chromosome.

In accordance with the diagnostic method of the present invention, the analysis of DLB would be as follows: a patient with suspected onset of dementia and/or with a non-definitive clinical-familial evaluation would be diagnosed by a genetic test determining the polymorphisms of the BChE gene described above. In the case of detecting the DLB specific genotypes, no additional tests or trial will be needed to diagnose correctly DLB. The direct application of genotyping represents an important save of money in the daily clinical practice.

The method of the invention is useful in the following suspected diagnosis: probable AD vs possible DLB; possible AD vs probable DLB; possible AD vs possible DLB; probable AD vs probable DLB; probable AD vs possible AD; possible DLB; and probable DLB. Physicians diagnose possible AD based on a full patient interview, covering personal and family medical history, combined with the outcome of any neurological, psychiatric, and lab tests conducted. Doctors are likely to expect AD when patient complains of a gradual progression of memory weakening, and when they are unable to find any other condition that could explain the memory loss. Doctors will be looking for disorders such as depression or hypothyroidism, neurological damage caused by stroke, or any medications that may be contributing to the loss of memory. An inability to uncover any contributory illness leads to the determination that AD is possible. Probable AD is a next step beyond possible Alzheimer's and means that a doctor is “relatively certain” that a patient has the disease.

Advantageously, the method of the invention allows a diagnosis of DLB without the need of obtaining samples by aggressive methods like a biopsy; and in this case a brain tissue microbiopsy. The method of the invention, being a genetic test, is performed on any biological sample removed from the subject, since it is applicable to any cell type of the body. In particular, blood, epithelial cells, and any other possible source of cell samples known in the art, may be used as sample within the method of the present invention.

In another embodiment, the determination of the genotype is carried out by one of the techniques selected from the group consisting of primer-specific PCR multiplex followed by detection, multiplex allele specific primer extension, a microarray-based method, and dynamic allele-specific hybridization. In a particular embodiment, it is carried out by primer-specific PCR multiplex followed by detection. Alternatively, individual PCR amplification reactions may be carried out for amplification of the different polymorphic sites and the genotype of the K variant.

The polymerase chain reaction (PCR) is the most widely used method for the in vitro amplification of nucleic acids. The PCR can be a real-time PCR, wherein the detection by labeled probes of the presence of the target genotypes is almost instantaneous to the amplification.

The amplification of the target polymorphisms can be performed by primer-specific PCR multiplex followed by detection by polyacrylamide electrophoresis, by analysis with a genetic analyzer, or by hybridisation with specific probes. Alternatively, various PCR reactions can be performed followed by agarose gel electrophoresis, by sequencing, or by hybridisation with specific probes. Preferably, specific probes may be immobilised in a microarray.

Determination of the genotype can be performed by Allele Specific Primer Extension (ASPE). This is a sequence specific enzymatic reaction technology that can be used to assay multiple SNPs in a single tube. The ASPE method involves two phases, an enzymatic reaction that determines the target genotype followed by a capture on solid microsphere surface for detection. Taking advantage of the solution phase kinetics, this technique allows sequence labeled microspheres to be used for detecting new templates. This is done with the help of an appropriate capture sequence attached to the allele specific oligonucleotide.

Optionally, detection may be carried out by DNA biochips/microarrays made with oligonucleotides deposited by any mechanism, by DNA biochips made, with oligonucleotides synthesized in situ by photolithography or any other mechanism. A microarray-based method that allow multiplex SNP genotyping in total human genomic DNA without the need for target amplification or complexity reduction can also be used for the genotyping of the BChE polymorphisms. This direct SNP genotyping methodology requires no enzymes and relies on the high sensitivity of the gold nanoparticle probes. Specificity is derived from two sequential oligonucleotide hybridizations to the target by allele-specific surface-immobilized capture probes and gene-specific oligonucleotide-functionalized gold nanoparticle probes. The assay format is simple, rapid and robust pointing to its suitability for multiplex SNP profiling at the ‘point of care’.

Furthermore, determination of the genotype can be performed by dynamic allele-specific hybridization (DASH), which represents the basis for throughput SNP genotyping in some laboratories. The core reaction principal of DASH is real-time (dynamic) tracking of allele-specific differences in the process of DNA denaturation. To achieve this, an oligonucleotide probe is first hybridized to the target DNA, a necessary component of essentially all genotyping methods. The target DNA comprises one strand of a PCR product immobilized onto a solid surface, and a single probe is used that is complementary to one of the target alleles. This assay concept was shown to be very precise (>99.9% accurate).

In a second aspect, the present invention provides a kit for carrying out the method as defined above, which comprises adequate means for determining the genotype of the polymorphisms in BChE gene.

In particular, the kit comprises primers which are capable of generating amplicons, said amplicons comprising the polymorphisms at positions 3687, 4206 and 4443 of SEQ ID NO: 1, and the polymorphism at position 934 of SEQ ID NO: 26. More particularly, the primers consist of SEQ ID NO: 8-19, as described in examples (Table 2). Using these primers, four amplicons are obtained which can be separated by size by capillary electrophoresis.

In a particular embodiment, the kit comprises adequate means for carrying out amplification by primer-specific PCR multiplex. Primers are labelled with different fluorophores which allow the identification of the four amplicons generated.

The kit provided by the present invention can be used in a routine clinical practice to identify patients that suffer from DLB, thus differentiating said patients from other patients that suffer from AD. With the kit of the invention the clinicians will be able to apply more individualized and risk-adapted treatment strategies to patients suffering from DLB.

In another aspect, the invention relates to the use of a kit as defined above, for the diagnosis of DLB.

The invention also refers to a method of determining whether a subject will respond to treatment with neuroleptics, by analyzing the genotype of the above mentioned polymorphisms in BChE gene. As the method allows determining whether a patient suffers from DLB or AD, is it possible to give the adequate treatment.

BChE overexpression is expected in DLB and therefore a usual treatment is the administration of cholinesterase inhibitors. In patients with elevated BChE levels, this treatment will be successful. On the contrary, patients carrying genotype combination AAAGCCK+ or AAAAC+KW will not respond to this treatment.

Throughout the description and claims the word “comprise” and variations of the word, such as “comprising”, are not intended to exclude other technical features, additives, components, or steps. Additional objects, advantages and features of the invention will become apparent to those skilled in the art upon examination of the description or may be learned by practice of the invention. Furthermore, the present invention covers all possible combinations of particular and preferred embodiments described herein. The following examples and drawings are provided by way of illustration, and are not intended to be limiting of the present invention.

### BRIEF DESCRIPTION OF THE DRAWINGS

FIG. 1. shows BChE expression levels in frontal cortex of DLB samples. Expression levels (EL) near 1, are similar to expression levels in controls as observed for the genotype combination AAAGCCK+. DLB brains carrying the KW genotype, as part of the genotype combination AAAAC+KW, show even lower BChE expression levels in the frontal cortex. All DLB brains that carried neither genotype combination AAAGCCK+ nor genotype combination AAAAC+KW overexpress BChE in the frontal cortex.

### EXAMPLES

### Post-Mortem Samples

Post-mortem frontal cortex samples with their clinical and neuropathological diagnosis were facilitated by the University of Barcelona Neurological Tissue Bank and the Bellvitge Institute of Neuropathology Brain Bank (BrainNet Europe) according to the established rules of the local ethic committees. They corresponded to 24 brains with common Lewy body disease (cLBD) (age at death: 79.9, age range from 64 to 90; female:male ratio 1.5:1), to 12 brains with pure dementia with Lewy bodies (pDLB) (age at death: 74.4, age range from 60 to 80; female:male ratio 1:2), to 26 AD brains (age at death: 78.1, age range from 61 to 95; female:male ratio 1:1.1) and 23 control brains (age at death: 68.5, age range from 54 to 83; female:male ratio 1:1.1).

Neuropathologic examination revealed that all AD brains presented AD Braak and Braak stage VI. Braak and Braak is a staging to evaluate/quantify AD in brain. It is used by neuropathologists to evaluate density of amyloid plaques and neurofibrillary tangles. AD stages following Braak and Braak, I-VI: neurofibrillary tangles; A-C: amyloid plaques. Two of the cLBD samples corresponded to Braak and Braak stage III, three to Braak and Braak stage 1V and the 19 remaining samples to stages V and VI. In pDLB brains Braak and Braak stages 0 to II were detected and in control samples AD related changes were absent. Whereas neither AD nor control brain showed PD-associated pathology, all pDLB as well as cLBD samples presented stages 5 and 6 corresponding to PD-related changes following classification of Braak and Braak.

### Clinically Diagnosed Samples

Blood samples were obtained from 223 AD patients (age: 71.1; age range from 49 to 86 years; female:male ratio 1:1.6) diagnosed in the Department of Neurology of our Hospital Germans Trias i Pujol, following NINCDS-ADRDA and DSM-IV criteria. Moreover, 59 age-matched control subjects (age: 68.8; age range from 46 to 91 years; female:male ratio 1:1.5).

In an additional experiment, a sample of 160 age-matched control subjects (age: 68.8; age range from 46 to 91 years; female:male ratio 1:1.5) was taken. The study was carried out after authorization of the Ethical Commitee from the Hospital and obtaining a signed informed consent.

### DNA Extraction

DNA from frozen brain samples was extracted by the use of the TRI Reagent following manufacturer's instructions. TRI Reagent solution combines phenol and guanidine thiocyanate in a monophasic solution and it is used for the consecutive extraction of RNA, DNA and proteins from the same sample.

After spectrophotometric determination of purity and concentration, DNA samples were stored at 4° C. until use. DNA extraction from blood was carried out by standard procedures based on DNA-binding on glass-filter membranes.

### BChE Promoter Sequencing

Since the BChE promoter sequence is constituted by approximately 5000 bp, three overlapping PCR fragments were amplified for their sequence analysis. PCR1 (primers BChEprom1UA and BChEprom1L, Table 1) yielded an 838 bp fragment spanning from position −1869 to position −1031. In PCR2 (primers BChEprom2UA and BChEpromS6, Table 1) a 837 bp fragment spanning from position −1152 to −315 and in PCR3 (primers BChEprom2UB and BChEprom2L; Table 1) a 688 bp fragment from position −473 to position +231 was obtained. PCR reactions with a final volume of 15 μl contained 1.7 mM MgCl2, 200 μM of each dNTP (Ecogen), 2 pmol of each primer, 1 unit EcoTaq DNA polymerase (Ecogen) and approximately 300 ng of DNA. Standard PCR programs with annealing temperatures of 58° C. for PCR1 and 60° C. for PCRs 2 and 3 were constituted by 30 cycles for PCR1 and 35 cycles for PCRs 2 and 3.

TABLE 1
Primers used for BChE promoter sequencing

| primer | primer sequence | |
|---|---|---|
| name | (5′->3′) | SEQ ID NO |
| BChEprom1UA | TGATAGGCTGACCGTATGCT | SEQ ID NO: 2 |
| BChEprom1L | ACCTCATCAGATGAGAAAGC | SEQ ID NO: 3 |
| BChEprom2UA | TCTCTTGGAAGCAGTTGACAT | SEQ ID NO: 4 |
| BChEpromS6 | CCATTATAGCTTCAATCTGTGC | SEQ ID NO: 5 |
| BChEprom2UB | AGATACATATCAGAGACATCCATT | SEQ ID NO: 6 |
| BChEprom2L | GAAGAGATCACTCTCATCCC | SEQ ID NO: 7 |

PCR products were purified by the use of the ExoSap-IT kit (GE Healthcare). Sequencing reactions were carried out with BigDye (BigDye™ Terminator vs 1.1 Cycle Sequencing Kit, Perkin Elmer), 10 pmol/μl of the respective primer and 3.5 μl of the purified PCR product. After cycle sequencing and DNA precipitation, the sequences were obtained on the ABI PRISMTM3100 (Perkin Elmer).

### Analysis of BChE Promoter Polymorphisms

Four new polymorphisms were found in the promoter region of the BChE gene. Three of them, as well as the well known K-variant polymorphism, were studied using mutation-specific-FOR (MS-PCR): A3687G, A4206G, C4443T and BChE-K. Each PCR reaction with a final volume of 15 μl contained 1.7 mM MgCl2, 200 μM of each dNTP (Ecogen), 2 pmol of each of the three primers (Table 2), 1 unit EcoTaq DNA polymerase (Ecogen) and 300 ng of DNA. Standard PCR programs of 35 cycles with annealing temperatures of 62° C. in the case of A3687G, BChE-K and of 57° C. in the case of A4206G, C4443T amplification were carried out. The obtained PCR fragments were separated on high resolution agarose gels. The A-allele of the BChE A3687G polymorphism was represented by a 153 bp and the G-allele by a 133 bp fragment. The K-allele was represented by a 149 bp fragment and the wildtype corresponding allele from the K-variant polymorphism, by a 169 bp band. A-allele of the BChE A4206G polymorphism was of 124 bp of length and the G-allele of 104 bp. Finally, in the case of the C4443T polymorphism, the T-allele corresponded to a 145 bp fragment and the C-allele to a 125 bp fragment.

TABLE 2
Primers used for BChE promoter genotyping

| Polymorp¹ | Primer name | primer sequence (5′->3′) | SEQ ID NO |
|---|---|---|---|
| A3687G | BChE-1314U | TCTTGAACTCCCAGACTGAAGCA | SEQ ID NO: 8 |
| | BChE-1314G | TACACAAAAGGTACAGAATACAC | SEQ ID NO: 9 |
| | BChE-1314A | TTATGTAATAACAAGTTAGTTACACAAAAG | SEQ ID NO: 10 |
| | | GTACAGAATACAT | |
| A4206G | BChE-795U | AAGTGCTCCACCTGCAAATATTA | SEQ ID NO: 11 |
| | BChE-795G | TAATCTTCTGTAAGTGATAGCC | SEQ ID NO: 12 |
| | BChE-795A | TTCTCAATGCAATATATTCTTAATCTTCTGT | SEQ ID NO: 13 |
| | | AAGTGATAGCT | |
| C4443T | BChE-558L | TGTCTCTGATATGTATCTCCTT | SEQ ID NO: 14 |
| | BChE-558CS | TCTTGACCAGAAAATTGTGGC | SEQ ID NO: 15 |
| | BChE-558TL | TATTCATTTTATTTTTCCTGTCTTGACCAGA | SEQ ID NO: 16 |
| | | AAATTTGTGGT | |
| BchE-K | BchE-4U | CTGTACTGTGTAGTTAGAGAAATTGGC | SEQ ID NO: 17 |
| | BchE-K | ATGGAATCCTGCTTTCCACTCCCATTCCGT | SEQ ID NO: 18 |
| | BchE-W | ATCATGTAATTGTTCCAGCGTAGGAATCCT | SEQ ID NO: 19 |
| | | GCTTTCCACTCCCATTCTCC | |

Polymorp¹: polymorphism name

### Statistical Analyses

Correspondence analysis (CORRESPONDENCE, Version 1.1, Data Theory Scaling System Group (DTSS), Faculty of Social and Behavioral Sciences, Leiden University, The Netherlands) permitted obtaining the correspondence table in the case of the neuropathologically diagnosed patient group. The distribution of the genotype combinations for both patient groups (neuropathologically and clinically diagnosed) was calculated by the SSPS version 11.0.

### Match of Clinical and Neuropathological Diagnosis

The match between both clinical and neuropathological diagnoses was first analyzed in the samples obtained from the Neurological Tissue Bank. Whereas 100% of AD patients coincided in their clinical and neuropathological diagnoses and 42% of pDLB patients received the diagnosis of DLB, only 17% of cDLB patients received the clinical diagnosis of DLB. Instead, 62% of them had been diagnosed as AD and 21% corresponded to other dementia related disorders. This observation fully correlates with the lack of diagnostic criteria for cDLB.

### Results

### Characterization and Disease Association of the BChE K-Variant

The BChE K-variant consist of a single nucleotide substitution from g to a at position 68974, where the g-allele is named W (wild type) and the a-allele K (mutated). An interesting finding of this analysis was the overrepresentation of K-allele carrying genotypes in cLBD but also in pDLD and AD when compared to controls (0.62 in cLBD, 0.42 in pDLB and 0.38 in AD vs. 0.13 in controls, p<0.001, p=0.090 and p=0.058, respectively). The further genotypic analysis revealed that the KW genotype presented similar frequencies in AD and controls, was slightly elevated in pDLB, but about one third of cLBD samples were KW-genotype carriers (Table 3). Whereas neither the H-nor the Jvariants were present in the studied samples, A-variant carrying genotypes were found at very low frequencies in the different diseases (0.04 cLBD, 0.08 in pDLB and 0.04 in AD vs 0 in controls; p=1, p=0.34 and p=1, respectively).

TABLE 3
Allele and genotype distribution of the BChE K-variant polymorphism
Table 3: Allele and genotype distribution
of the BChE K-variant polymorphism

Genotype frequencies

| Disease | n¹ | WW | KW | KK | p² |
|---|---|---|---|---|---|
| cLBD | 24 | 0.38 | 0.29 | 0.33 | 0.003 |
| pDLB | 12 | 0.58 | 0.17 | 0.25 | 0.145 |
| AD | 26 | 0.62 | 0.08 | 0.30 | 0.047 |
| C | 23 | 0.87 | 0.09 | 0.04 | |

¹n: sample number;
²p: Exact test p value for genotypic comparisons between each disease and controls.

### Characterization and Disease Association of BChE Promoter Polymorphisms

The three BChE promoter polymorphisms were single nucleotide changes: at position 3687, where A was changed by G; A was substituted by G at position 4206 and C to T at position 4443.

To ascertain if the polymorphisms showed a disease-specific association, allelic and genotypic frequencies for the promoter polymorphisms were determined in neuropathologically diagnosed brain samples including cLBD, pDLB, AD and controls. First, the polymorphisms were analyzed independently and then, the existence of genotype combination was also tested.

The study of the A3687G polymorphism revealed an approximately three-fold increase of the AA genotype in AD when compared to cLBD, pDLB and controls (0.54 in AD vs. 0.21 in cLBD, p=0.152; 0.16 in pDLB, p=0.298 and 0.13 in controls, p<0.001). In contrast G-allele carrying genotypes corresponding to the A4206G polymorphism were accumulated in cLBD, pDLB, as well as AD in comparison with controls (0.33 in cLBD, 0.17 in pDLB and 0.23 in AD vs. 0.04 in controls, p=0.023, p=0.262 and p=0.105, respectively). Although the accumulation of G-allele carrying genotypes was not disease specific, this accumulation seems to be of certain importance since G-allele carrying genotypes were almost absent in controls. The CC-genotype corresponding to the C4443T polymorphism was present at a very low frequency in pDLB when compared with cLBD as well as controls. Conversely, the frequency of the TC-genotype was elevated almost two fold in both pDLB and cLBD in comparison with AD and it was also significantly higher than in controls.

### Analysis of Genotype Combinations

### Correspondence Analysis

Genotype combinations (GenComb) resulting from three BChE promoter polymorphisms: (1) 1314AA at position 3687 (polymorphism: A3687G), (2) 795AG at position 4206 (polymorphism: A4206G), (3) 558CC at position 4443 (polymorphism: C4443T), and BChE-K (KW or KK en la posición 68974 (common polymorphism KW in exon4), were studied by correspondence analysis. The representation of the results in a correspondence table (Table 4) allowed the easy detection of disease-specific genotype combinations.

TABLE 4
Correspondence table of BChE genotype
combinations in a neuropathological
sample including three disease and a
control group.

| | | AD | pDLB | cDLB | Controls |
|---|---|---|---|---|---|
| 1 | AAAACCKK | 2 | 0 | 0 | 0 |
| 2 | AAAACCKW | 0 | 0 | 1 | 1 |
| 3 | AAAACCWW | 0 | 1 | 0 | 0 |
| 4 | AAAATCWW | 0 | 0 | 0 | 2 |
| 5 | AAAATCKK | 0 | 1 | 0 | 0 |
| 6 | AAAATCWW | 1 | 0 | 0 | 0 |
| 7 | AAAATTKK | 1 | 0 | 0 | 0 |
| 8 | AAAATTWW | 5 | 0 | 0 | 0 |
| 9 | AAAGCCKK | 0 | 0 | 3 | 0 |
| 10 | AAAGCCKW | 0 | 0 | 1 | 0 |
| 11 | AAAGTTKK | 1 | 0 | 0 | 0 |
| 12 | AGAACCKK | 0 | 0 | 0 | 1 |
| 13 | AGAACCWW | 0 | 0 | 0 | 1 |
| 14 | AGAATCKK | 2 | 0 | 2 | 0 |
| 15 | AGAATCKW | 0 | 1 | 2 | 0 |
| 16 | AGAATCWW | 0 | 2 | 2 | 5 |
| 17 | AGAATTKW | 0 | 0 | 1 | 0 |
| 18 | AGAATTWW | 5 | 1 | 3 | 5 |
| 19 | AGAGTCKK | 2 | 1 | 3 | 0 |
| 20 | AGAGTCKW | 2 | 0 | 1 | 0 |
| 21 | GGAACCWW | 1 | 0 | 0 | 0 |
| 22 | GGAATCWW | 0 | 0 | 1 | 2 |
| 23 | GGAATTKW | 0 | 1 | 1 | 0 |
| 24 | GGAATTWW | 3 | 3 | 3 | 5 |
| 25 | GGAGCCKW | 0 | 0 | 0 | 1 |
| 26 | GGAGTCKK | 0 | 1 | 0 | 0 |
| 27 | GGAGTTWW | 1 | 0 | 0 | 0 |
| | | 26 | 12 | 24 | 23 |

### Common Genotype Combinations

The first, overall analysis revealed the presence of 27 different GenComb (Table 3). Since most of them (59%) were present in one or two samples only, their frequency was very low (0.01 and 0.02). Both most frequent GenComb (N° 18 and 24), represented 32.9% of the whole sample and were present at similar frequencies in all groups.

### Disease-Specific Genotype Combinations

When analyzed by diseases, two important disease-specific GenComb could be detected. On one hand, the GenComb AAAATTWW was only present in AD samples with a relative high frequency of 0.19.

When combining genotype combinations 9 and 10 and defining them as the common GenComb AAAGCCK+, this GenComb was the most frequent (0.17) disease-specific GenComb found in LBD.

### Analysis of Genotype Combinations in the Clinical Sample

To confirm the data obtained by the study of the post-mortem sample, a clinical sample composed of a group of 223 AD patients and a group of 160 control individuals was also studied. The AD patients had been diagnosed between 1998 and 2002, but since the latest guidelines for clinical DLB diagnosis had been established in 2005, it can be expected that between 20 and 40% of these AD patients should be misdiagnosed DLB patients.

### Correspondence Analysis

The distribution of the resulting GenComb is shown in a correspondence table (Table 5). Taking into account that the GenComb was constituted by 4 polymorphisms, it was very surprisingly to find only 25 different GenComb in that sample constituted by 383 individuals (Table 5). 63.6% of all detected GenComb coincided in both samples.

TABLE 5
Correspondence table of BChE genotype
combinations in a clinical sample
including an Alzheimer disease and a
control group.

| | | AD | C |
|---|---|---|---|
| 1 | AAAACCKK | 1 | 0 |
| 2 | AAAACCKW | 7 | 2 |
| 3 | AAAACCWW | 2 | 3 |
| 4 | AAAATCKW | 4 | 0 |
| 5 | AAAATCWW | 5 | 1 |
| 6 | AAAATTKK | 1 | 0 |
| 7 | AAAATTWW | 5 | 5 |
| 8 | AAAGCCKK | 6 | 2 |
| 9 | AAAGCCKW | 5 | 1 |
| 10 | AAAGCCWW | 0 | 1 |
| 11 | AAAGTCKW | 4 | 6 |
| 12 | AGAACCKK | 0 | 1 |
| 13 | AGAACCKW | 2 | 2 |
| 14 | AGAACCWW | 0 | 1 |
| 15 | AGAATCKK | 0 | 1 |
| 16 | AGAATCKW | 24 | 27 |
| 17 | AGAATCWW | 24 | 20 |
| 18 | AGAATTKW | 2 | 1 |
| 19 | AGAATTWW | 32 | 25 |
| 20 | AGAGTCKW | 19 | 8 |
| 21 | AGAGTCWW | 0 | 1 |
| 22 | AGAGTTKW | 1 | 0 |
| 23 | AGAGTTWW | 1 | 0 |
| 24 | GGAATTKW | 2 | 4 |
| 25 | GGAATTWW | 76 | 49 |
| | | 223 | 161 |

### Common Genotype Combinations

Three of the four most frequent GenComb were the same in both samples: combination 25 (24 in Table 4) with a frequency of 0.33 vs. 0.16 in the post-mortem sample, combination 19 (18 in Table 4) with a frequency of 0.15 vs. 0.16 in the post-mortem sample, 17 (16 in Table 4) with a frequency of 0.11 vs. 0.11 in the post-mortem sample and combination 16 (15 in Table 4) with a frequency of 0.13 vs. 0.03 in the post-mortem sample (Table 5).

### Disease-Specific Genotype Combinations

When analyzing the distribution of the GenComb detected as DLB- and AD-specific in the post-mortem sample, GenComb AAAATTWW was detected in only 2% of AD patients, but also in 3.1% of control individuals (Table 5). These frequencies indicated that AAAATTWW is not suitable to be considered as a disease marker.

GenComb AAAGCCK+ was found in with a frequency of 0.05 in the AD and of 0.02 in the control group (Table 5).

Taking into account that the AD patients had been clinically diagnosed about 8 years ago, much before the establishment of the new guidelines for DLB diagnosis, the clinical histories of the 11 patients carrying the GenComb AAAGCCK+ were revised. All 11 presented at least one of the symptoms compatible with DLB, corroborating AAAGCCK+ as a possible DLB marker.

When furthermore taking into account that 20-40% of the AD patient group could be misdiagnosed DLB patients, the disease-specific frequency of AAAGCCK+ increases and would range between 15-30%. The specificity of AAAGCCK+ was of 98.1% and the sensitivity between 15 and 30%.

### Genotype Combination Dependent on Relative BChE Expression

To detect possible specific features for DLB-AAAGCCK+-carriers, BChE expression levels in frontal cortices of 22 DLB samples in comparison with 13 AD and 12 control samples were determined.

### RNA Isolation and Reverse Transcription

TRI-Reagent (MRC, Cincinnati, USA) was used for RNA isolation according to the manufacturer's protocol. Briefly, 100 mg tissue samples were homogenized in a 1.5 ml tube with a sterile piston in 1.0 ml of TRI-Reagent. Homogenates were incubated 5 min at room temperature and then centrifuged at 12,000 g for 10 min at 4° C. to pellet insoluble material and highmolecular-weight DNA. After phase separation, RNA was precipitated with isopropanol and resuspended in an appropriate volume of DEPC-treated water. RNA quantity was determined spectrophotometrically at A260, RNA purity was ascertained from optical density ratio at 260 nm and 280 nm. RNA integrity was ascertained by the use of the Agilent 2100 Bioanalyzer (Agilent Technologies, Santa Clara, USA). Only samples with RIN values higher than 6 were stored at −80° C. until use.

First-strand cDNA synthesis was carried out using Ready-to-go™ You-Prime First-Strand Beads (Amersham Pharmacia Biotech, Uppsala, Sweden). Two mg of RNA were incubated with random hexamers and the First-Strand Beads at 37° C. during 1 hour. The resulting cDNA was either immediately used for PCR or stored at −20° C. until use.

### Real Time PCR

The relative expression of BChE mRNA was determined using a Rotor-Gene 6000 (Corbett Life Science, Sydney, Australia). A QuantiTect SYBR Green PCR Kit (QiaGen, Hilden, Germany) was used to minimize the primer-dimer content. Fifteen ml reactions further contained 16 pmol of each primer (BChE 2U GAGTAGATCCATAGTGAAACGG, SEQ ID NO: 20, and BChE 6LRNA CAGCGATGGAATCCTGCTTT, SEQ ID NO: 21) and 1 ml of cDNA. To study relative BChE amounts, two housekeeping genes were also analyzed, betaactin (primers: beta-actin U2 TCTACAATGAGCTGCGTGTG, SEQ ID NO: 22, and beta-actin L3 TAGATGGGCACAGTGTGGGT, SEQ ID NO: 23) and betaglucuronidase (GUS; primers: GUS-U1 ATGTGGTTGGAGAGCTCATT, SEQ ID NO: 24 and GUS-L2 TGTCTCTGCCGAGTGAAGAT, SEQ ID NO: 25) (M. Barrachina et al., “TaqMan PCR assay in the control of RNA normalization in human post-mortem brain tissue”, Neurochem Int 2006, vol. 49, pp. 276-84).

After a 15-minutes-denaturation step, followed by 30 seconds of annealing at 56° C. for all BChE, GUS and beta-actin, end fluorescence data were acquired during a standard 72° C. extension. A final melting analysis was run for all products to determine the specific amplification. Relative expression data were achieved by the deltadelta Ct method based on the assumption of similar PCR efficiencies to analyze relative gene expression (T. D. Schmittgen et al., “Analyzing real-time PCR data by the comparative C(T) method”, Nat Protoc 2008, vol. 3, pp 1101-8). Therefore, different primer pairs of each gene and isoform were tested to obtain fragments with a length between 100 and 150 base pairs that become amplified with similar efficiencies. Since PCR efficiencies can vary in each run, a standard curve was included in each and not only in the initial run. Only runs with similar efficiencies together with a correct standard curve (R>0.99 and RA2>0.99) were suitable for further analyses. Standard curves were generated by amplifying the same serially diluted cDNA control sample. All assays were performed twice and independently to assure their reproducibility and minimize possible errors, including additionally a negative control in each run

### Results

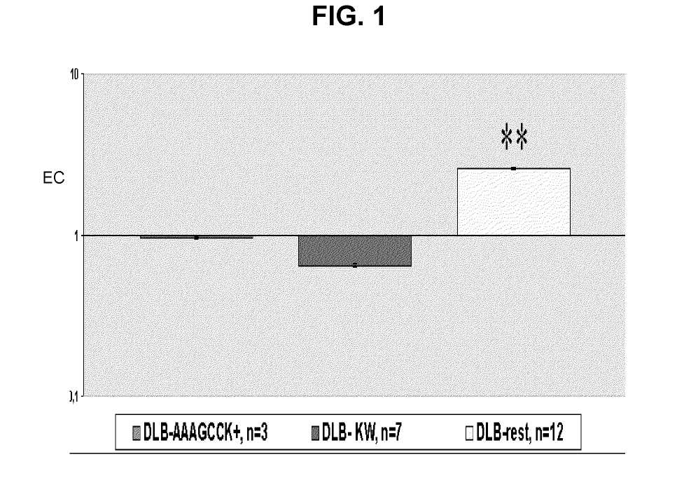

Main relative expression analysis revealed a slight, but not significant increase of BChE expression in DLB: 1.53 (1.13-2.07), but not in AD: 1.26 (1.17-1.36), when compared to controls. Especially DLB cases showed a wide range of variance estimates. When analyzing BChE expression in dependence on GenComb, it was found that DLB-AAAGCCK+-carriers (n=3) and control samples presented similar BChE expression levels (FIG. 1). DLB samples with the KW genotype (n=7) showed also BChE expression levels similar to controls.

In contrast, the rest of all DLB samples (n=12) exhibited marked BChE overexpression (FIG. 1). It is important to mention that ranges of variance estimates were very low in all three DLB groups, the group of AAAGCCK+-carriers, the group of KW-carriers and the group of remaining samples.

Since it has been described that DLB is characterized by an even higher cholinergic deficit than AD, it can be expected that BChE expression is increased especially in DLB. In fact, the present study reveals, that about 60% of all DLB patients exhibit a more than three-fold BChE overexpression. Instead, the rest of patients are carriers of BChE genotype combinations/genotypes associated to lower BChE expression levels (FIG. 1).

### Identification of a Second DLB-Specific BChE-Genotype Combination

Due to the results of BChE expression analyses, all genotype combinations were reanalyzed in the clinical sample. Carriers of the KW genotype were found with similar frequencies in both the AD and control groups. Instead, GenComb AAAAC+KW, was present in 11 patients of the AD group (frequency of 0.05) and in 2 individuals of the control group (frequency of 0.012). The revision of the clinical histories also revealed in all of them symptoms compatible with DLB.

Similar to GenComb AAAGCCK+, AAAAC+KW-frequency would range between 15-30%, taken into account that 20-40% of our AD group, are actually DLB patients. The specificity of AAAAC+77 KW would be of 98.7% and its sensitivity between 15 and 30%.

If then combining both GenComb, the testing of BChE genotype combinations AAAGCCK+ and AAAAC+KW would allow the detection of 30-60% of DLB cases, with a specificity of 96.8%.

### REFERENCES CITED IN THE DESCRIPTION

I. G. McKeith, “Consensus guidelines for the clinical and pathologic diagnosis of dementia with Lewy bodies (DLB): report of the Consortium on DLB International Workshop”, J. Alzheimer's Dis. 2006, vol. 9, pp. 417-23
F. Parmo-Folloni et al., “Two new mutations of the human BCHE gene (IVS3-14T>C and L574fsX576)” Chemico-Biological Interactions 2008, vol. 175, pp. 135-7
A. B. Singleton et al., “Butyrylcholinesterase K: an association with dementia with Lewy bodies”, Lancet 1998, vol. 351, pp. 1818.
R. Lane et al., “BuChE-K and APOE epsilon4 allele frequencies in Lewy body dementias, and influence of genotype and hyperhomocysteinemia on cognitive decline”, Mov. Disord. 2009, vol. 24, pp. 392-400.
W. Maetzler et al., “No differences of butyrylcholinesterase protein activity and allele frequency in Lewy body diseases” Neurobiol. Dis. 2009, vol. 35, pp. 296-301
M. Barrachina et al., “TaqMan PCR assay in the control of RNA normalization in human post-mortem brain tissue”, Neurochem Int 2006, vol. 49, pp. 276-84
T. D. Schmittgen et al., “Analyzing real-time PCR data by the comparative C(T) method”, Nat Protoc 2008, 5 vol. 3, pp 1101-8

1. An in vitro method for the diagnosis of dementia with Lewy bodies comprising:
determining in a biological sample from a subject, the genotype of the following polymorphisms in butyrylcholinesterase (BChE) gene:
the polymorphic site at position 3687 in NCBI Accession Number NG_{—}009031 (i.e. SEQ ID NO: 1),
the polymorphic site at position 4206 in SEQ ID NO: 1,
the polymorphic site at position 4443 in SEQ ID NO: 1, and
the polymorphic site at position 68974 in NCBI Accession Number NG_{—}009031 (i.e. position 934 in SEQ ID NO: 26).

2. The method according to claim 1, wherein the genotype is:
adenine for both alleles at position 3687,
an adenine for one allele and a guanine for the other allele at position 4206,
cytosine for both alleles at position 4443, and
an adenine for one allele at position 68974,
being this genotype indicative of dementia with Lewy bodies and distinguishing from Alzheimer disease.

3. The method according to claim 1, wherein the genotype is:
an adenine for both alleles at position 3687,
an adenine for both alleles at position 4206,
cytosine for one allele at position 4443, and
an adenine for one allele and a guanine for the other allele at position 68974;
being this genotype indicative of dementia with Lewy bodies and distinguishing from Alzheimer disease.

4. The method according to claim 1, wherein the determination of the genotype is carried out by one of the techniques selected from the group consisting of individual PCR amplification reactions, primer-specific PCR multiplex followed by detection, multiplex allele specific primer extension, a microarray-based method, and dynamic allele-specific hybridization.

5. The method according to claim 4, wherein the determination is carried out by amplification by primer-specific PCR multiplex followed by detection.

6. The method according to claim 5, wherein the detection is carried out by hybridization with specific probes

7. The method according to claim 6 wherein the specific probes are immobilized in a microarray.

8. The method according to claim 1, wherein the biological sample is a blood sample.

9. The method according to claim 1, wherein the biological sample is an epithelial cell sample.

10. A kit for carrying out the method as defined in claim 1, which comprises adequate means for determining the genotype of the polymorphisms in BChE gene.

11. The kit according to claim 8, which comprises primers which are capable of generating amplicons, said amplicons comprising the polymorphisms at positions 3687, 4206 and 4443 of SEQ ID NO: 1, and the polymorphism at position 934 of SEQ ID NO:26.

12. The kit according to claim 11, wherein the primers consist of SEQ ID NO:8-19.

13. The kit according to claim 10, wherein the primers are labelled with fluorophores and the kit comprises reagents for performing a primer-specific PCR mutliplex.

14. (canceled)
